(19) United States
(12) Patent Application Publication
(10) Pub. No.: US 2010/0049029 A1
(43) Pub. Date: Feb. 25, 2010

(54) PIEZOELECTRIC MAGNETIC RESONANCE ELASTOGRAPH (MRE) DRIVER SYSTEM

(75) Inventors: Geng Li, Hong Kong (CN); Veng-Vai V. Lam, Hong Kong (CN); Chih Lin I, Hong Kong (CN); Edward S. Yang, Hong Kong (CN)

Correspondence Address: FULBRIGHT & JAWORSKI L.L.P, 2200 ROSS AVENUE, SUITE 2800, DALLAS, TX, 75201-2784, US

(73) Assignee: Hong Kong Applied Science and Technology Research Institute Co., Ltd., Shatin (CN)

(21) Appl. No.: 12/194,949

(22) Filed: Aug. 20, 2008

(51) Int. Cl. A61B 5/055

(52) U.S. Cl. 600/410

(57) ABSTRACT

An array of two or more piezoelectric drivers generates shear waves in a region of interest of a human undergoing a MRE test. The use of the array of drivers allows for better diagnosis of disease of the humans or animals.

### TECHNICAL FIELD

This application relates in general to magnetic resonance elastographic systems, and in specific to systems and methods that use an array of piezoelectric drivers in magnetic resonance elastographic systems.

### BACKGROUND

Magnetic Resonance Elastography (MRE) is an MRI-based method for imaging the mechanical properties of tissue. The technique is used to depict the spatial distribution of tension in skeletal muscle, brain tissue, breast tissue, liver tissue, prostate tissue, etc. In this technique, a driver, e.g. pneumatic or electromechanical driver, is used to generate shear waves in a region of interest, such as brain, breast, liver, prostate, etc. of a human subject, while the human subject is located in a magnetic resonance imaging (MRI) system. In some instances, shear waves are generated by applying mechanical motion to the surface of the region of interest of the human subject. A mechanical actuator is coupled to the human subject, and provides cyclic motion that is synchronized to the MRI imaging sequence. Another way to generate shear waves in the tissue is to use a piezoelectric bending element. In other instances, a needle is inserted into the tissue of the animal or human subject, and the waves are generated by vibrating the needle. For more information about piezoelectric drivers, see Chan, Q. C. C. et al., “Localized Application of Shear Waves to Tissues for MR Elastography via a Needle Device,” Proceedings of the 13^{th} ISMRM, Florida, USA May 7-13, 2005; Chan, C. C., et al., “Shear Waves Induced by Moving Needle in MR Elastography, Proceedings of the 26^{th} Annual International Conference of the IEEE EMBS, San Francisco, Calif. USA, Sep. 1-5, 2004, pg. 1-3; Chan, Q. C. C., et al. “Needle Shear Wave Driver for Magnetic Resonance Elastography,” Magnetic Resonance in Medicine 55:1175-1179 (2006); Chen, Jun, et al., “Imaging Mechanical Shear Waves Induced by Piezoelectric Ceramics in Magnetic Resonance Elastography,” http://scholar.ilib.cn/Abstract.aspx?A=kxtb-e200606016, (downloaded Jun. 19, 2008); the disclosures of which are hereby incorporated herein by reference.

### BRIEF SUMMARY

Various embodiments as described herein may be used to improve the operations of MRE systems. Devices, systems, and methods described herein may lead to improved medical care of humans and also animals. Embodiments of the invention involve the use of an array of two or more piezoelectric drivers to generate shear waves in a region of interest of a human subject undergoing a MRE test.

One embodiment of the invention involves a phased array driver for a magnetic resonance elastography system comprising: a first driver having a piezoelectric element that comprises a MRI compatible piezoelectric material; a second driver having a piezoelectric element that comprises a MRI compatible piezoelectric material; wherein the first driver and the second driver are arrayed to produce share waves in a region of interest of a human subject.

Another embodiment of the invention involves a phased array driver for a magnetic resonance elastography system comprising: a first driver having a piezoelectric element that comprises a PVF2 material; a second driver having a piezoelectric element that comprises a PVF2 material, wherein the first driver and the second driver are arrayed to produce shear waves in a region of interest of a human subject.

Another embodiment of the invention involves a magnetic resonance elastography system comprising: a magnetic resonance imaging (MRI) system that scans a subject; and a phased array of drivers that produce shear waves in a region of the subject from a signal, wherein each of the drivers in the array comprises a piezoelectric element having a MRI compatible piezoelectric material or PVF2 material.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 depicts an exemplary arrangement for an MRE system, according to embodiments of the invention;

FIG. 2 depicts exemplary results of a test using the system of FIG. 1;

FIG. 3 depicts another exemplary result of another test using the system of FIG. 1;

FIGS. 4A-4C depict the components of an exemplary driver, according to embodiments of the invention;

FIGS. 5A-5B depict the components of another exemplary driver, according to embodiments of the invention;

FIG. 6 depicts the components of another exemplary driver, according to embodiments of the invention;

FIG. 7 depicts the components of another exemplary driver, according to embodiments of the invention;

FIGS. 8A-8D depict a comparison of the shear waves generated by a single driver and the shear wave generated by a phase array of two drivers, according to embodiments of the invention;

FIGS. 9A and 9B depict exemplary arrangements of phase array drivers, according to embodiments of the invention;

FIGS. 10A and 10B depict exemplary arrangements of phase array drivers located on a patient, according to embodiments of the invention; and

FIG. 11 depicts another exemplary arrangement for an MRE system, according to embodiments of the invention.

### DETAILED DESCRIPTION

Embodiments of the invention use one or more driver arrays to induce an oscillating stress to produce shear waves that propagate through a human to allow tissue and/or organs to be imaged. The shear waves alter the phase of the magnetic resonance signals produced by a MRI system, and from the altered phase, mechanical properties of the subject can be determined, such as the elasticity, viscosity of the tissue or organ, the density of the tissue or organ, and the size and/or shape of tissue or organ. Note that multiple tests conducted at different times, can provide changes in elasticity, density, viscosity, size, and shape over time to detect diseases at a very early stage. The information provided by MRE test(s) can be used by a practitioner, along with data from other sources, e.g. x-ray test, CT tests, ultrasound tests, PET tests, regular MRI tests, chemical tests (e.g. blood tests, etc.), to provide a more accurate diagnosis of a disease or illness of a patient at a very early stage.

Data that includes the mechanical properties of the subject can allow for earlier diagnosis of diseases with increased specificity and sensitivity. The earlier and more accurate the diagnosis, the better chance of recovery for the patient. Diseases that benefit from having mechanical property data include brain diseases such as Alzheimer's disease and mild cognitive impairments, liver diseases such as cirrhosis, spleen diseases, kidney diseases such as kidney stones or tumors, pancreas diseases such as tumors, prostate diseases such as prostate carcinoma, uterine diseases such as uterine tumors, and arterial diseases such as arteriosclerosis and the like. For example, liver cirrhosis may manifest itself as a change in elasticity of the liver tissue, but not show any change in liver chemistry. Thus, detecting a change in the elasticity may lead to an earlier diagnosis and treatment of liver disease. As another example, Alzheimer disease manifests itself as a change in elasticity and density of the brain, which can be readily detected at an early stage by a MRE test. Other tests can also detect Alzheimer disease at an earlier stage, e.g. a PET scan test, however a PET scan uses radiation, which is detrimental to a patient. Any disease that manifests itself as a change in the mechanical properties of tissue or organs can be detected using embodiments of the invention.

In some applications, the production of shear waves in the tissues can be accomplished by physically vibrating the surface of the subject with a pneumatic or an electromechanical device. For example, shear waves may be produced in the breast or liver or prostate by direct contact with the oscillatory driver to the surface of the human body. Also, with organs like the liver or breast, the oscillatory force can be directly applied by means of an applicator that is inserted into the organ by a needle driver. However, if possible, it is preferential to apply the force noninvasively, i.e. to the surface of the subject.

The driver may comprise a piezoelectric device, which vibrates to produce the shear waves. One type is a piezoelectric material that is made especially for MRI applications. Materials for nonmagnetic bending actuators are made by Piezo Systems, Inc., 186 Massachusetts Avenue, Cambridge, Mass. 02139. Another type of piezoelectric device is uses a polyvinylidene fluoride (PVF2) membrane as the vibrating surface. Such a material is known as Pro-Wav, which is available from S. Square Enterprise Company Limited, Pro-Wave Electronics Corporation. One advantage of using the PVF2 material is that the membrane is not brittle, and is capable of conforming to different curved surfaces of the body of the patient. This provides a more accurate reading, by allowing full contact with the body of the patient, and thus better insertion of the shear waves. Another advantage of using piezoelectric drivers is that the size for the drivers are much smaller than the other types of drivers, e.g. pneumatic drivers, and thus allow for easier set up. Also the piezoelectric drivers do not suffer the power attenuation that pneumatic drivers experience, namely the air tube loses power rapidly over distance. Another advantage is that the piezoelectric drivers do not use coils which are susceptible to MRI induced eddy currents, which can produce artifacts in the images.

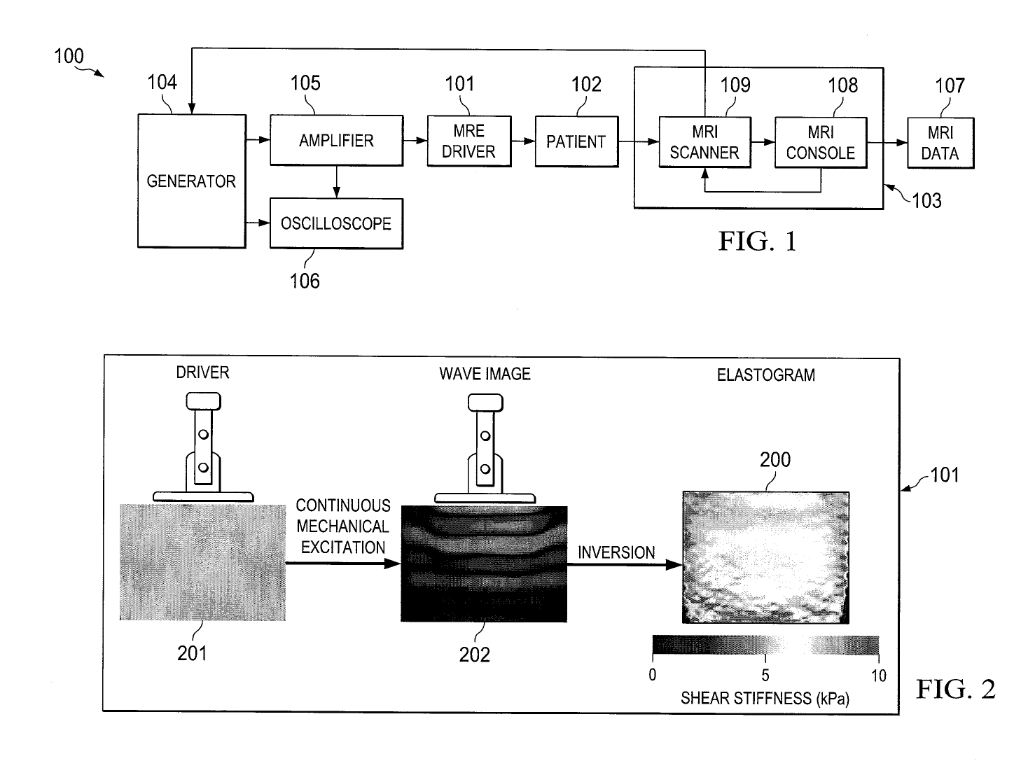

FIG. 1 depicts an exemplary arrangement for an MRE system 100, according to embodiments of the invention. System 100 includes a MRE driver 101, which is a piezoelectric driver that comprises a MRI compatible piezoelectric material or a PVF2 membrane. The driver 101 is placed in contact with patient 102, which may be a normal subject. The patient 102 with the driver is then placed into a MRI system 103, which comprises a MRI scanner 109. The MRI scanner 109 is controlled by MRI console 108. The operation of the MRE system 100 produces MRE data 107, which may be graphically viewed on a display device, not shown. The MRE driver 101 uses a signal that is produced by generator 104, and is amplified by amplifier 105. The oscilloscope 106 monitors the signal from the generator 104. The signal generation of generator 104 is synchronized with the operation of the MRI system 103. A trigger on the MRI scanner provides a signal to the generator to initiate a vibration. For example, the signal activates the generator to form ten pulses at its set frequency.

FIG. 2 depicts exemplary results 200 of a test using the system of FIG. 1. In FIG. 2, the driver 101 is located on the surface of any interested region 201 of patient 102. The driver 101 is vibrated to produce shear waves 202 in the tissue region 201. The resulting data 200 depicts an image that shows the differences in elasticity of the region 201. The image is formed by analysis of the MRE data produced by the test. The wave image is inverted to produce the elastogram image of the resulting data 200.

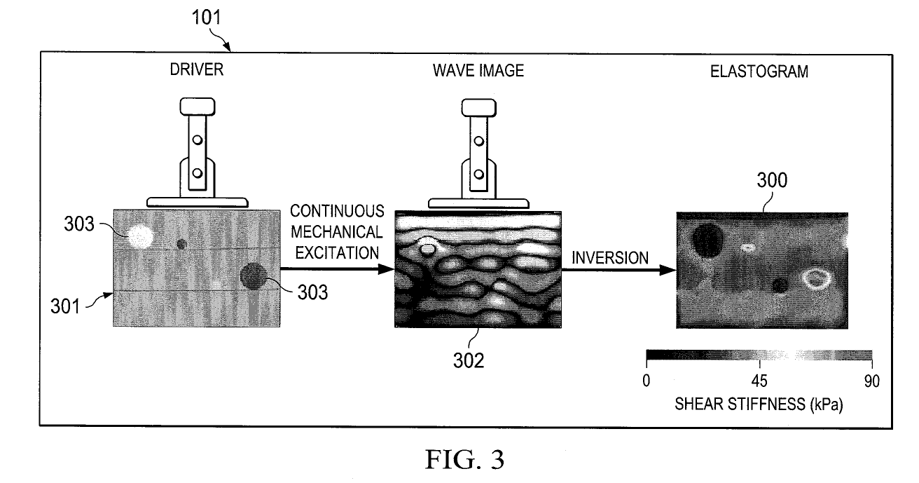

FIG. 3 depicts another exemplary results 300 of another test using the system of FIG. 1. In FIG. 3, the driver 101 is located over tissue/organ region 301 of patient 102. In this example, the tissue region 301 includes tumors 303ab. The driver 101 is vibrated to produce shear waves 302 in the tissue region 201. The resulting data 300 depicts an image that shows elasticity of the region 201. As the shear wave passes through a tumor 303a, which is softer or more elastic than the surrounding region, the wave becomes shorter. As the shear wave passes through a tumor 303b, which is harder or less elastic than the surrounding region, the wave becomes longer. Note that the tumors have different elasticity values than the surrounding regions, and thus are readily identifiable.

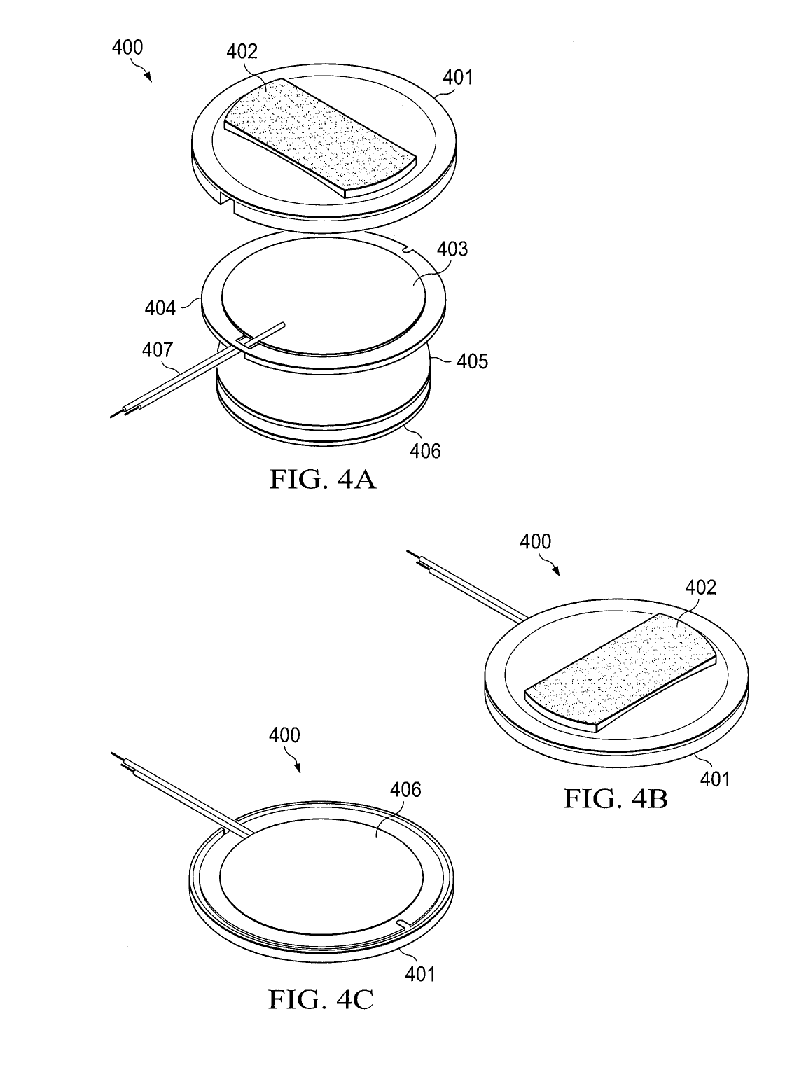

FIGS. 4A-4B depict the components of an exemplary driver, according to embodiments of the invention. FIG. 4A depicts an exploded perspective view showing the different components of the driver. FIG. 4B depicts a perspective view of the driver showing the side that is placed onto the patient. FIG. 4C depicts a perspective view of the driver showing the side that faces away from the patient. The driver 400 includes a housing 401 that includes a fixing device 402 that connects the driver to the patient. The fixing device 402 may be Velcro™, an adhesive, a snap, or the like. The driver 400 includes mounting frame 403 that supports the piezoelectric element 404. The element 404 may comprise a piezoelectric device composed of special made MRI compatible piezoelectric material or PVF2. The driver 400 also includes reinforcement layer 405 that supports and protects the piezoelectric element 400, and insulation layer 406 to prevent electric current from the piezoelectric material traveling to the patient. The driver operates by receiving electricity through wires 407. This embodiment is useful for tests involving breasts, heart, abdominal organs such as the liver, the spleen, the pancreas, a kidney, the prostate, as well as the pelvis.

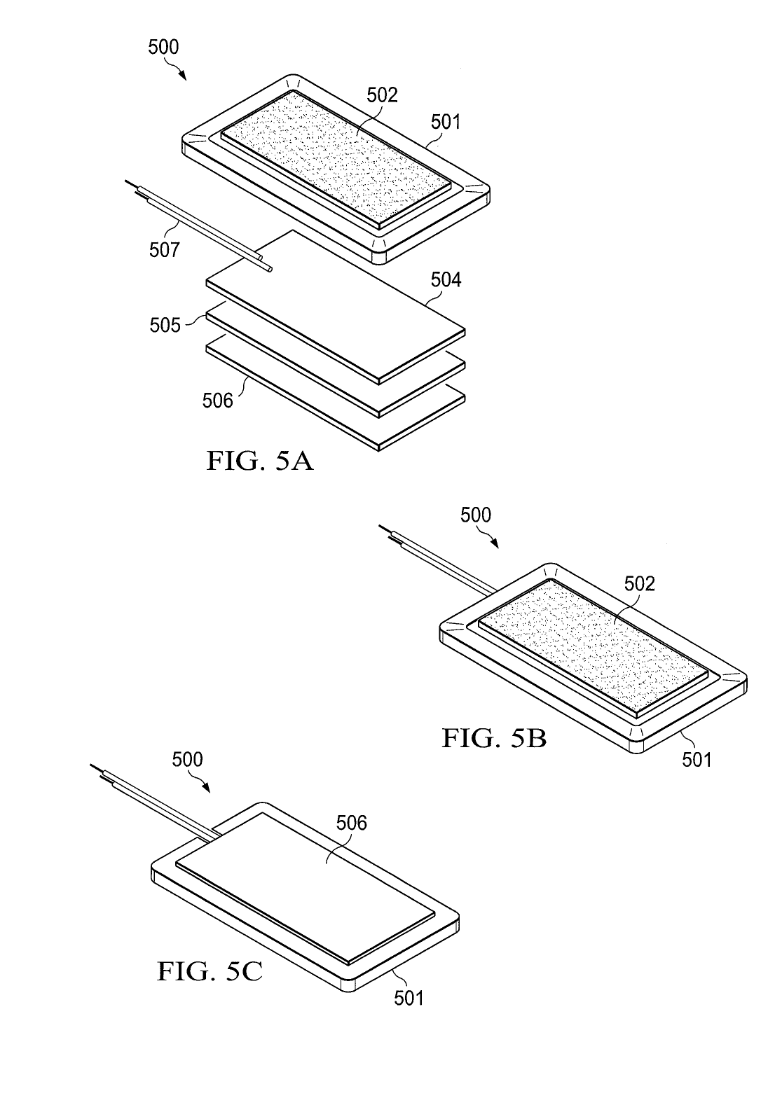

FIGS. 5A-5B depict the components of another exemplary driver, according to embodiments of the invention. FIG. 5A depicts an exploded perspective view showing the different components of the driver. FIG. 5B depicts a perspective view of the driver showing the side that is placed onto the patient. FIG. 5C depicts a perspective view of the driver showing the side that faces away from the patient. The driver 500 includes a housing 501 that includes a fixing device 502 that connects the driver to the patient. The fixing device 502 may be Velcro™, an adhesive, a snap, or the like. The driver 500 includes the piezoelectric element 504. The element 504 may comprise a piezoelectric device composed of a special made MRE compatible material or PVF2. The driver 500 also includes reinforcement layer 505 that supports and protects the piezoelectric element 500, and insulation layer 506 to prevent the electric current from the piezoelectric material traveling to the patient. The driver operates by receiving electricity through wires 507. This embodiment is useful for tests involving the head, neck, and extremities.

FIG. 6 depicts the components of another exemplary driver, according to embodiments of the invention. The driver 600 includes a flexible housing 601 that includes a fixing device 602 that connects the driver to the patient. The fixing device 602 may be Velcro™, an adhesive, a snap, or the like. The driver 600 includes a PVF2 piezoelectric element within the housing. The driver operates by receiving electricity through wires 603. This embodiment is useful for tests involving the arms or legs.

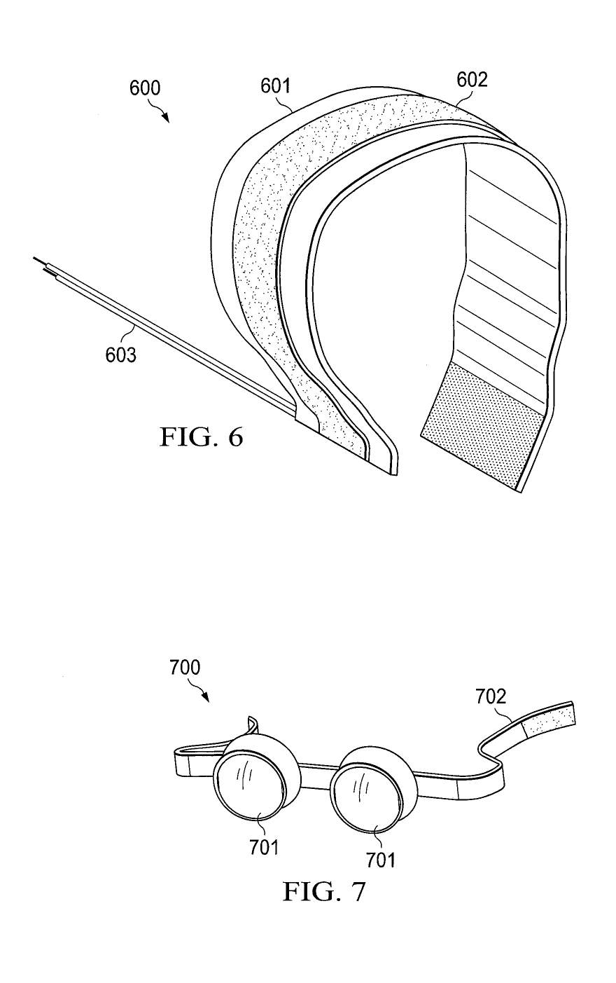

FIG. 7 depicts the components of another exemplary driver, according to embodiments of the invention. The driver 700 is adapted to be used in tests involving breasts. The driver includes a flexible housing 701 that includes a fixing device 702 that connects the driver to the patient. The fixing device 702 may be Velcro™, an adhesive, a snap, or the like. The driver 700 includes two PVF2 piezoelectric elements within the housing thus allowing both breasts to be examined at the same time.

The size of the drivers may be varied as needed. Some regions of a patient's body may require a larger vibration, and hence a larger driver, to produce the shear waves needed to examine the region. Some portions may be thicker or comprise tissue that is more attenuating than other regions. For example, the human brain is encased in the skull, which comprises a thick bone material. The shear waves are greatly attenuated by the skull. Thus, the vibration power needed to analyze the brain should be larger. Other regions, e.g. arms and legs, are thinner and therefore, a lower vibration power can be used. Typically, the deeper the region of interest, the greater the power should be.

One embodiment of a MRE system can use a plurality of drivers in a phased array. A plurality of drivers would be located at various sites on the patient. The sites are selected according to the anatomic location of the human body to minimize interference between the waves created by the drivers and to illuminate the region of interest (ROI) wholly. The drivers may comprise MRI compatible piezoelectric materials or PVF2 material. Using a phased array of drivers increases the sensitivity of the MRE test and reduces the effects of attenuation. To reduce the wave interference induced by having multiple drivers, each driver is synchronized with the same frequency, and the same power, and triggers at the same time.

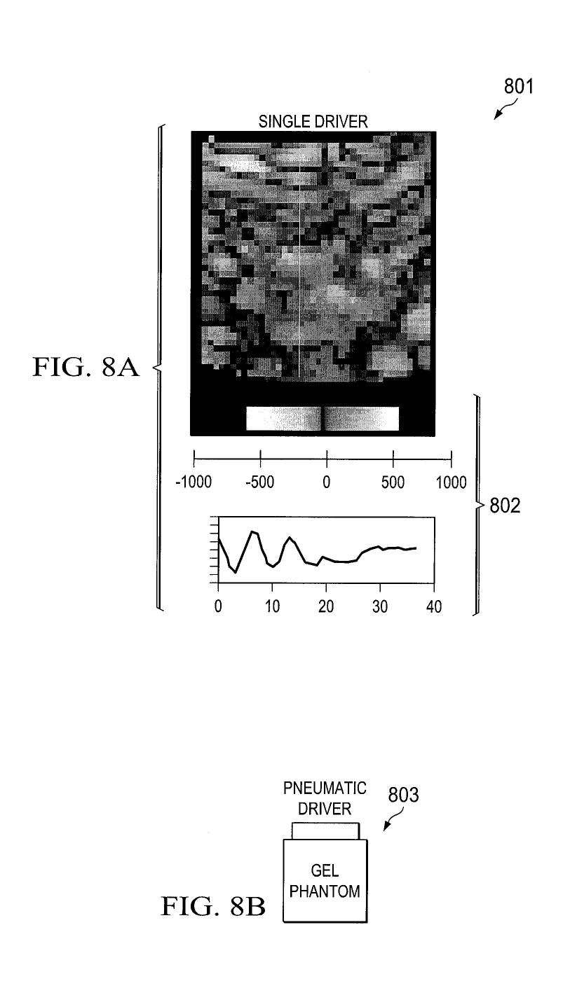

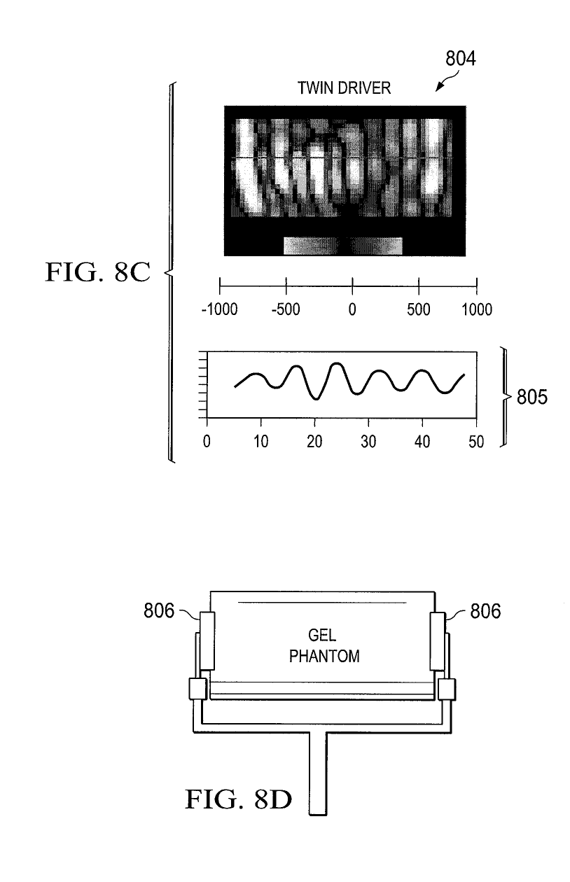

FIGS. 8A-8D depict a comparison of the shear waves generated by a single driver and the shear wave generated by a phased array of two drivers, according to embodiments of the invention. In FIG. 8A, a single driver is used to produce the shear waves as shown. The driver 803 is arranged on the tissue as shown in FIG. 8B. The waves produced are relatively strong near the surface, but are rapidly attenuated as the distance increases from the driver, as shown in the diagram 802. In FIG. 8C, an array of two drivers is used to produce the shear waves 804 as shown. The drivers 806 are arranged around the tissue as shown in FIG. 8D. The waves produced appear to be relatively unattenuated throughout the sample, as shown in the diagram 805. The drivers trigger at the same time, with the same power, and the same frequency, and have symmetrical locations so the shear waves constructively interfere with each other to form a stronger signal.

Tests conducted on regions of the body that are relatively deep or include attenuating tissue benefit by using a phased array. The pluralities of drivers allow the shear wave to penetrate to the deeper areas, and pass through attenuating materials. The drivers of the area may be located in areas that have less attenuating materials than other regions. For example, some locations of the skull attenuate less than other areas. Knowledge of human anatomy and physiology will allow for proper placement.

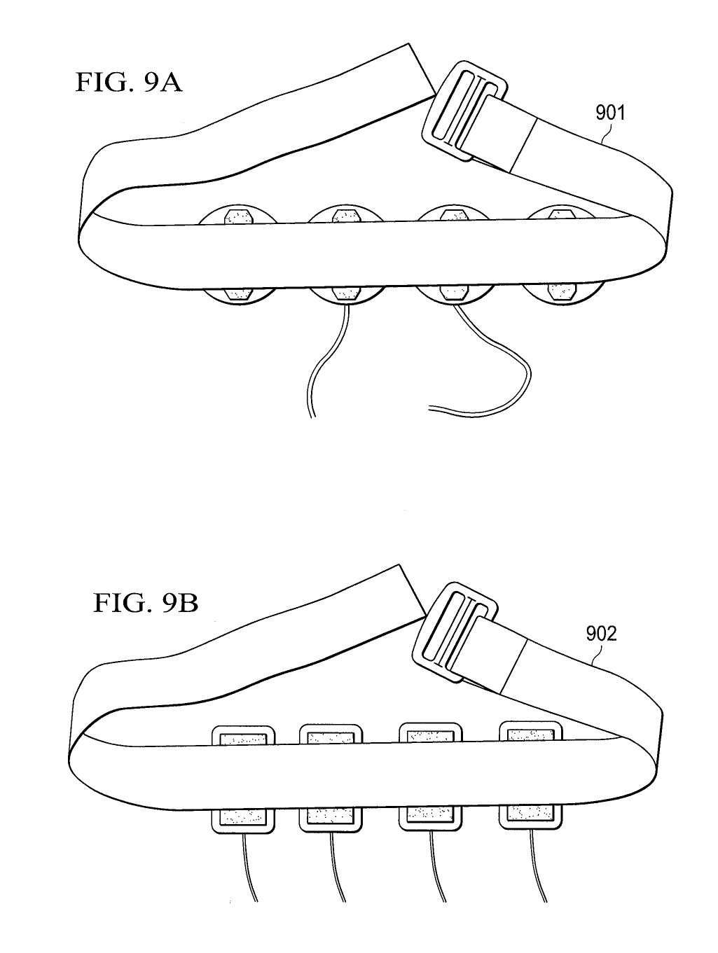

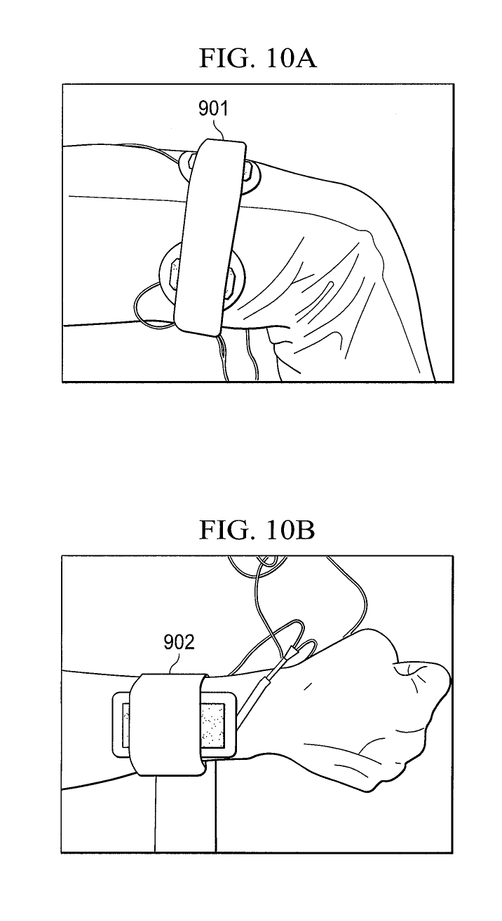

FIGS. 9A and 9B depict exemplary arrangements of phase array drivers, according to embodiments of the invention. FIG. 9A depicts a plurality of drivers 400 of FIGS. 4A-4C. FIG. 9B depicts a plurality of drivers 500 of FIGS. 5A-5C. In each embodiment, the drivers are located on a belt that may be secured to a patient. In FIG. 9A, only two of the four drivers will be used in a test, so the wires of the other two are disconnected. In FIG. 9B, all four drivers are to be used, and thus all four drivers have power wires. Note that the number of drivers is by way of example only as two or more drivers may be used to form the array. Note that each driver may be shaped differently from the other drivers to accommodate different shapes, sizes and contours of patient.

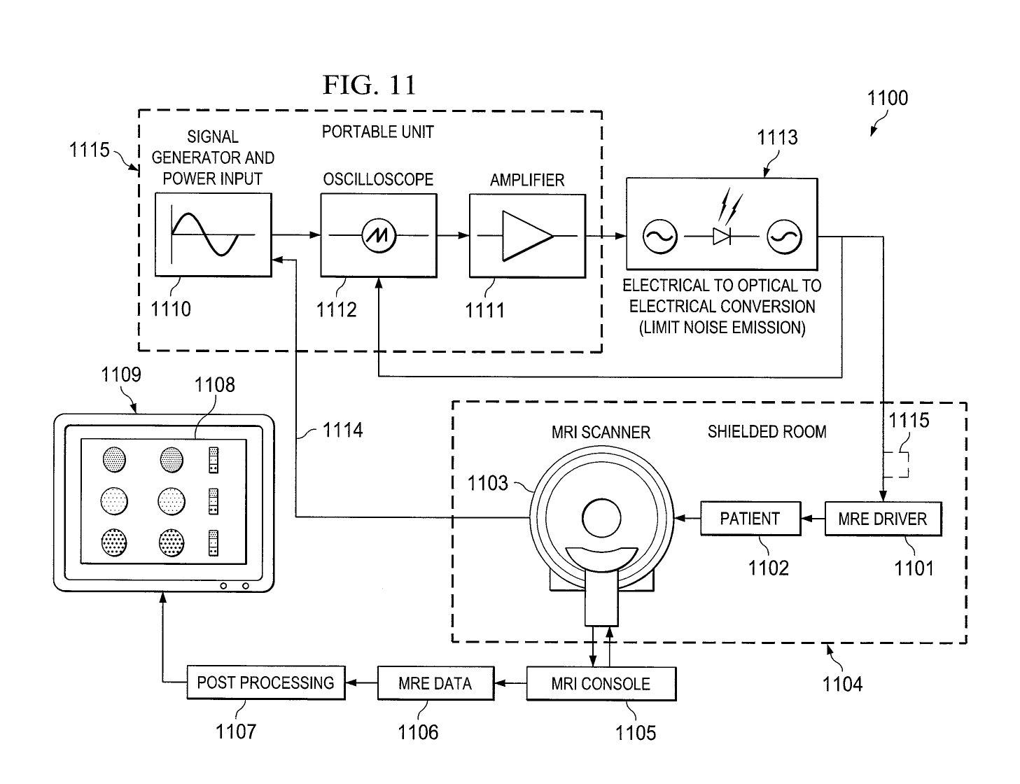

FIGS. 10A and 10B depict exemplary arrangements of phase array drivers located on a patient, according to embodiments of the invention. FIG. 11A depicts the array of FIG. 10A being used on a patient. In this example, all four of the drivers are being used, and thus all four have wires to receive power. FIG. 11B depicts the array of FIG. 10B being used on a patient. In this example, only two drivers are used because the arm is small relative to other regions of the body. Note that each driver may be shaped differently from the other drivers to accommodate different shapes, sizes and contours of patient.

FIG. 11 depicts another exemplary arrangement for an MRE system, according to embodiments of the invention. System 1100 includes a MRE driver 1101, which is a piezoelectric driver that comprises a MRE compatible piezoelectric material or membrane. The driver 1101 is placed in contact with patient 1102, which may be a normal subject. The patient 1102 with the driver is then placed into a MRI scanner 1103. The patient 1102 with the driver 1101 and the MRI scanner 1103 are located in a shielded room 1104. The MRI scanner 1103 is controlled by MRI console 1105. The operation the MRE system 1100 produces MRE data 1106, which is processed by post-processing software 1107 to produce images 1108 that may be graphically viewed on a display device 1109.

The MRE driver 1101 uses a signal that is produced by generator 1110, and is amplified by amplifier 1111. The oscilloscope 1112 displays the signal from the generator 1110. The signal generation of generator 1110 is synchronized with the operation of the MRI system 1103 by signal 1114. Typical frequencies are 60 Hz, 80 Hz, 100 Hz, or 150 Hz. The signal duration lasts through the MRE scan.

This arrangement also includes an electrical-optical-electrical conversion. The driver 1101 requires an electric signal to operate. However, using metal wire to provide the signal may induce interference in the signal, because the metal wire will inductively receive EM fields generated by the MRI scanner 1103. Thus, the scanner 1103 can interfere with the operation of the driver 1101. The signal leaving the amplifier 1111 is converted to an optical signal by converter 1113. Such a conversion may be accomplished by using an LED or an LED laser. The light signal is then carried on a fiber optic line to the driver 1101. Another converter 1115 that is proximate to the driver 1101 converts the light signal back into an electrical signal. The second converter may be located next to the driver 1101 or may be integrated with the driver 1101. The second converter may also comprise an amplifier to boost the electric signal that is being sent to the driver. The amplifier may be instead of or in addition to amplifier 1111. Note that in this arrangement, the generator 1110, the oscilloscope 1112, and the amplifier 1111 comprise a single component 1115 that may be portable.

Note that in this embodiment, the MRI scanner 1103 controls the activation of the signal generation 1110. However, the MRI console 1105 gives the command to the MRI scanner 1103 to control signal generator 1110. The generator can be controlled to change the frequency of all or some of the drivers. Thus, each of the drivers can receive the same signal frequency or may receive different signal frequencies. Note that the drivers may receive the signal frequency at the same time to have the same phase or may receive the signal at different times to have different phase.

Additionally, the amplifier 1111 can be controlled to change the power of the signal being sent to all or some of the drivers. The power can be increased to all or some of the drivers. Thus, the drivers may all be operating at the same power level or may have different power levels.

Note that each of the drivers in the array may be the same size or may have different sizes. Furthermore, a smaller driver located in one region may receive more power than a larger driver located in another region. Thus, the shear wave produced by the drivers may have similar wave power, because the smaller driver is receiving more power.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A phased array driver for a magnetic resonance elastograph system comprising:
a first driver having a piezoelectric element;
a second driver having a piezoelectric element;
wherein the first driver and the second driver are arranged to produce shear waves in a subject.

2. The driver of claim 1, wherein each piezoelectric element comprises one of a MRI compatible material and a PVF2 material.

3. The driver of claim 1, wherein a shape of the first element is different than a shape of the second element.

4. The driver of claim 1, further comprising:
a converter that receives an optical signal and converts the optical signal into an electrical signal for use by the first driver and the second driver.

5. The driver of claim 1, wherein the subject is a human.

6. The driver of claim 5, wherein the driver array is used to diagnosis a medical condition of the human subject.

7. The driver of claim 1, wherein the phase array driver operates according to a driver signal, wherein the driver signal is produced by a signal component that comprises:
a signal generator that forms the signal;
an oscilloscope that displays the signal and facilitates monitoring of the signal; and
an amplifier that increases power of the signal.

8. The driver of claim 7, wherein the signal component comprises a single container.

9. The driver of claim 7, further comprising:
a converter that converts the electrical signal to an optical signal.

10. The driver of claim 1, further comprising:
a connector that secures the driver array to the subject.

11. A magnetic resonance elastrography system comprising:
a magnetic resonance imaging (MRI) system that scans a subject; and
a phased array of drivers that produce shear waves in a region of the subject from a signal, wherein each of the drivers in the array comprises a piezoelectric element.

12. The system of claim 11, wherein an operation of the array of drivers is controlled by the MRI system.

13. The system of claim 11, further comprising:
a signal component that generates a signal that operates the phased array.

14. The system of claim 13, wherein the signal component comprises:
a signal generator that forms the signal;
an oscilloscope that displays the signal and facilitates monitoring of the signal; and
an amplifier that increases power of the signal.

15. The system of claim 14, wherein the signal component comprises a single container.

16. The system of claim 13, further comprising:
an fiber optic wire that is connected between the signal component and the phased array.

17. The system of claim 16, wherein the signal component further comprises:
a converter that converts an electrical signal from the signal component into a light signal.

18. The system of claim 16, wherein the phased array further comprises:
a converter that converts a light signal from the fiber optic wire into an electrical signal that is useable by the phased array.

19. The driver of claim 11, wherein the subject is a human.

20. The driver of claim 19, wherein the driver array is used to diagnose a medical condition of the human subject.

21 The device of claim 1, wherein the drivers operate to vibrate at a frequency, wherein the frequency is selected from 60 Hz, 80 Hz, 100 Hz, and 150 Hz.

22. The system of claim 11, wherein each piezoelectric element comprises one of a MRI compatible material and a PVF2 material.
